(19) United States
(12) Patent Application Publication
(10) Pub. No.: US 2017/0007546 A1
(43) Pub. Date: Jan. 12, 2017

(54) MICROPARTICLES WITH EFFICIENT BIOACTIVE MOLECULE INCORPORATION

(71) Applicant: KONINKLIJKE PHILIPS N.V., Eindhoven (NL)

(72) Inventors: Caecilia Hendrina Theodora Chlon, Eindhoven (NL); Marce Rene BOHMER, Eindhoven (NL)

(21) Appl. No.: 15/275,808

(22) Filed: Sep. 26, 2016

Related U.S. Application Data
(63) Continuation of application No. 13/387,503, filed on Jan. 27, 2012, now abandoned.

(30) Foreign Application Priority Data
Jul. 31, 2009 (EP) 09166962.2

(51) Int. Cl. A61K 9/50; A61K 9/00; A61K 31/337; A61K 41/00

(52) U.S. Cl. CPC A61K 9/5031; A61K 9/5089; A61K 41/0028; A61K 9/0009; A61K 31/337

(57) ABSTRACT

The present invention relates to, drug-filled polymeric microparticles comprising a gas core and shell that combine high incorporation efficiency for hydrophilic and/or hydrophobic drugs with a large, preferably hollow, core.

### CROSS REFERENCE TO RELATED APPLICATION

This is a continuation of prior application Ser. No. 13/387,503 filed Jan. 27, 2012.

### FIELD OF THE INVENTION

The invention relates to a method for the preparation of drug filled polymer microparticles comprising a gas core and shell, which particles are suitable as part of a therapeutic composition, especially for drug delivery.

### BACKGROUND OF THE INVENTION

Microparticle based ultrasound contrast agents are in use to enhance ultrasound contrast in medical imaging. Recent research demonstrates that they have therapeutic potential for drug delivery from the vasculature as well; microbubbles can increase the permeability of the endothelium and therefore lower the barrier for drug delivery from the vasculature. Drug delivery can also take place directly from drug loaded microbubbles themselves, which would allow a drastic change in the biodistribution with the potential to reduce side-effects of, for instance, cytotoxic agents.

U.S. Pat. No. 6,896,659 relates to a method of delivering a therapeutic agent to a localized region within a subject using ultrasound to trigger the release of the agent from hollow microbubbles having a specified set of mechanical properties. The agents disclosed in U.S. Pat. No. 6,896,659 have a controlled fragility which is characterized by a uniform wall thickness to diameter ratio that defines discrete threshold power intensity. U.S. Pat. No. 6,896,659 specifically discloses a single emulsion method for preparing the microbubbles wherein cyclooctane is used as a liquid forming the core in the creation of the microbubbles. This cyclooctane is in a later step removed by lyophilization.

The incorporation of drugs in polymeric spheres via a double emulsion methods is known from the prior art. (Sonsoles Diez et al, European Journal of Pharmaceutics and Biopharmaceutics 63 (2006) 188-197) (Diez et al.)

According to the double emulsion method described herein, polymeric spheres are synthesized by preparing a first emulsion by adding an aqueous drug containing solution into a polymer solution in an organic solvent. This first emulsion is subsequently emulsified again in an aqueous phase, after which the organic solvent is extracted.

### SUMMARY OF THE INVENTION

It is desirable to synthesize an agent with a single large gaseous core that can be acoustically activated at a pressure and frequency acceptable in ultrasound drug delivery, in combination with the capacity of this agent to comprise hydrophilic and/or hydrophopic drugs.

We have surprisingly found that the amount of incorporation of both hydrophobic and hydrophilic drugs is increased while obtaining a stable microparticle with a large hollow core by using specific ratios of polymer and solvents.

Therefore the invention in a first aspect relates to the following method for preparing biologically active agent filled polymer microparticles, said method comprising the steps of:

providing a first emulsion (A) by mixing an organic solvent (1), a biodegradable polyester, and an organic non-solvent for the polymer (2), wherein the ratio biodegradable polyester/organic non solvent is 1:10 to 1:1, and adding to this mixture from 0 to 40% v/v of an aqueous solution and wherein a biologically active agent is added to the organic mixture and or aqueous solution

preparing a second emulsion (B) by adding to this first emulsion (A) excess of an aqueous solution

applying conditions for volatizing the organic solvent (1)

applying conditions for removal of water

applying conditions for removing of the non-solvent (2).

By using this method, polymeric microparticles are obtained that combine high incorporation efficiency for hydrophilic and/or hydrophobic drugs with a large, preferably hollow, core. Microparticles formed via the method according to Diez et al lead to polymer spheres with a density higher than that of water, which thus can be centrifuged in the bottom of a vial. This implies that there is no large core present. This large core however, is essential for acoustic properties that can be used for the actual drug release via ultrasound. By performing the method according to the invention, small polymeric microparticles are obtained in a size range from 0.5-5 micrometers, more specifically from 1-3 micrometers that have a single gaseous core and are stable upon redispersion.

The biologically active agent is added in step a) to the organic solvent in the case of hydropbobic agents and in the aqeous phase for hydrophilic agents.

In a further aspect the invention relates to particles obtained by this method, their inclusion in contrast agents and therapeutic agents and to contrast agents or therapeutic compositions wherein the majority of particles can be activated by ultrasonic power that has an intensity in a range that is usual for ultrasound diagnostic imaging.

Particular and preferred aspects of the invention are set out in the accompanying independent and dependent claims. Features from the dependent claims may be combined with features of the independent claims and with features of other dependent claims as appropriate and not merely as explicitly set out in the claims.

### BRIEF DESCRIPTION OF THE DRAWINGS

FIG. 1-7: particle size distributions of microparticles obtained via the method according to the invention.

### DETAILED DESCRIPTION OF THE EMBODIMENTS

In the context of the invention the following definitions are used.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

### Method According to the Invention

### Step a)

In the method according to the invention step (a) comprises providing a mixture comprising a shell forming polymer, a first solvent (1) and a second non-solvent (2).

This mixture is preferably made at a temperature between from 4 to 30° C., more preferred around room temperature.

In the context of the invention, the shell forming polymer is a biodegradable polyester, more preferably a biodegradable polyester selected from the group comprising polylactide either the L or the DL form, poly-lactide-co-glycolide and polycaprolacton, a combination thereof and block co-polymers thereof. Hollow polymer microparticles are obtained using biodegradable polyesters with a molecular weight range of 1.000 and 200.000 g/mol. More preferably, the molecular weight of the biodegradeble polyester is between 1500 and 20.000 and even more preferably between 1500 and 5000.

In a preferred embodiment, the biodegradable polyester comprises at least one moiety modified with at least one hydrophobic group that is preferably selected from the group comprising fluoride, alkyl chain comprising from 6 to 24 carbon atoms or a combination of these.

In the context of the invention solvent (1) is preferably a good solvent for the shell forming polymer. It is preferred that solvent (1) is a good solvent for the polymer forming the shell and non-solvent (2) is a bad solvent for the polymer forming the shell.

Solvent (1) preferably dissolves in water to at least some extent. Solvent (1) is preferably relatively volatile.

Solvent (1) is preferably a solvent having a vapor pressure higher than water under the conditions of step (c), more preferably selected from the group comprising dichloromethane, dichloroethane or chloroform are examples of solvents that can be used, but also non-chlorinated solvents such as isopropylacetate can be used.

It is believed that non-solvent (2) is present to make particles comprising a gaseous core and a shell instead of solid particles. Therefore suitable compositions for solvent (2) are desirably relatively non volatile compositions wherein the chosen shell composition does not dissolve or only to a very low extent. Contrary to solvent 1 for non-solvent (2) it is highly preferred that the solubility in water is very low to zero.

Non-solvent (2) is selected from the group comprising organic compositions that have a vapor pressure significantly lower than water under the conditions of step (d). More preferred the vapor pressure of non-solvent (2) is at least 2 times lower, more preferably 4 times lower, than that of water under the conditions of step (d). The non-solvent (2) is selected such that its vapor pressure is still sufficiently high to enable removal under freeze-drying conditions optionally in combination with a suitable reduced-pressure that can easily be reached using well-known standard equipment.

This low vapor pressure and the low solubility will ensure that solvent (2) really stays inside the capsule being formed, leading in the end to form a capsule with a hollow gaseous space. Preferably the capsule comprises at least one hollow space. Most preferred the capsule comprises one main hollow space. If non-solvent (2) is disappearing from the capsule before the removal of solvent 1 is complete, the particles will show too much shrinking, thereby increasing their wall thickness, in step (c).

In a preferred embodiment non-solvent (2) is selected from the group comprising hydrocarbons having a carbon chain length of from 10 to 20 carbon atoms. It was found that it is advantageous to select the non-solvent (2) from cyclooctane, cyclodecane, decane, camphor or a combination thereof. In a most preferred embodiment, non-solvent (2) comprises cyclooctane, even more preferred the non-solvent (2) essentially consists of cyclooctane. In the context of the invention “essentially consists of” means that at least 80 wt %, preferably 90 to 100 wt % of the non-solvent (2) is cyclooctane.

Optionally in step (a) pre-mixtures are used of solvent (1) and (2) and of the shell composition and solvent (1).

Added to the mixture of organic solvent (1) and (2) and of the shell forming polymer is 0 to 0.4, more preferably approximately 0.2 volumes of aqueous solution, resulting in emulsion A. Preferably, this aqueous solution is buffered.

To create an emulsion, preferably stirring or another form of agitation/shear forces is applied. Optionally further emulsification treatment is included to form an emulsion with the desired, preferably monodispersed, particle size distribution. Suitable equipment to obtain such emulsification treatment is for example selected from colloid mills, homogenizers, sonicaters.

Optionally the emulsion either before or after such treatments, is pressed through a glass filter. When desired such treatment may be repeated multiple times.

If apart from a gas phase a nonpolar liquid reservoir is desired in the microbubble, the organic solvent or non solvent can be mixed with an oil or alkane that cannot or with much more difficulty be freeze-dried out, for instance hexadecane.

Hydrophobic therapeutic compositions can be included in the core via this non-polar liquid reservoir. Hexadecane or paraffin oils may be used to solubilize a therapeutic composition in the core. Potential drugs that may be included in the particle core include anti-cancer drugs such as paclitaxel. We have surprisingly found that hexadecane is a very suitable carrier liquid for hydrophobic therapeutical compositions. We have found that such compositions easily stay dissolved or finely dispersed in hexadecane and these compositions will therefore incorporate inside the core of the particles in a remaining oil phase. Therefore the dissolved composition is released from the particles only after activation with ultrasound. Therefore in a preferred embodiment, the invention relates to the claimed particles further comprising at least one carrier liquid for a therapeutical composition. The most preferred carrier liquid is hexadecane.

Hydrophilic drugs are added to the first aqueous solution in emulsion A.

### Step b)

A further step (b) comprises combining the emulsion of step (a) with an aqueous composition, thereby forming an emulsion B of the mixture of step (a) in an aqueous phase.

Preferably the shell composition containing mixture of step (a) is added to an aqueous composition. To create an emulsion, preferably stirring or another form of agitation/shear forces is applied.

Optionally further emulsification treatment is included to form an emulsion with the desired, preferably monodispersed, particle size distribution.

Suitable equipment to obtain such emulsification treatment is for example selected from colloid mills, homogenizers, sonicaters.

Optionally the emulsion either before or after such treatments, is pressed through a glass filter. When desired such treatment may be repeated multiple times.

An alternative embodiment to create the desired particle size with a narrow distribution is using methods that produce monodisperse emulsions such as inkjet technology and emulsification using substrates with microchannels or micropores.

It is highly preferred that the conditions are controlled such that water and, especially, non-solvent (2) are not yet removed.

Optionally in step (a) or (b) a stabilizing composition is included. Such stabilizing composition is preferably selected from the group of surfactants and polymers comprising for example polyvinyl alcohol, albumin or a combination of at least two surfactants and/or polymers. If such stabilizing agent is included in the process, the process preferably includes a washing step after removal of solvent (1) to remove the stabilizer. The stabilizer is preferably used in a concentration between 0.1-20%, more preferably between 5-15%.

### Step c)

The conditions in step (c) are preferably such that the majority of non-solvent (2) is not yet removed, more preferred essentially no non-solvent (2) is removed. Hence it is preferred that in this step no measures are taken to reduce the pressure around the mixture such as by applying a vacuum.

A suitable way to remove solvent (1) is to increase the temperature for example to a temperature to a value a few degrees below the boiling point of the solvent to be removed-, or simply by stirring the mixture for a given amount of time.

Without wishing to be bound by any theory it is believed that whilst the solvent (1) vaporizes the concentration of the shell composition in the emulsion internal phase increases to over the solubility threshold and at such moment in time the shell composition will start to precipitate.

This precipitation then leads to the formation of a shell of polymer at the surface of the emulsion inner phase (emulsion droplet). It is believed that once the majority or all of solvent (1) has vaporized, a shell composition results which covers a core comprising non-solvent (2), water and optionally other ingredients that may have been added at an earlier stage of the process.

### Step d)

In this step, the microparticles are isolated from the aqueous phase and optionally washed to purify the particles. Separation of the particles can easily be facilitated by for example centrifugation, as the microparticles have a density that is lower than that of water.

### Step e)

In a further step (e) conditions are applied to remove water from the core. This is immediately followed by the removal of non-solvent (2) in step (f).

It is highly preferred that the removal of water and non-solvent (2) are separated in two different steps. In practice it may be unavoidable to have some overlap between these steps but overlap should preferably be avoided. Generally removal of water is obtained e.g. by freeze-drying techniques. Removal of non-solvent (2) may require further reduction of pressure.

The particles that result after step (e) are usually re-suspended in a suitable liquid before use. If the agent is to be used as a contrast agent or therapeutic agent for animals or humans, it is preferred that the particles are re-suspended in an aqueous physiological salt solution.

### Polymeric Particle Obtained by the Method According to the Invention

A preferred aspect of the invention relates to a polymeric particle comprising a gas core and a polymeric shell wherein the particle has an average particle size of 0.5 to 5 micrometer. More preferably, at least 90% of the particles has a particle size of 0.5 to 5 micrometer, even more preferable more than 95% of the particles has a particle size of 0.5 to 5 micrometer.

Such particles can be acoustically activated by application of ultrasound at a mechanical index of at most 3, more preferred at most 1.6, more preferred at most 1.2, even more preferred at most 1.0, even more preferred at most 0.8.

It is preferred that the activation sets off at a mechanical index above 0.2, more preferred between 0.2 and 0.8, even more preferred at a lower limit of between 0.2 and 0.6.

For ultrasound mediated drug release applications it is desired that the polymeric microparticles are re-suspended in a suitable liquid forming a dispersion.

Most preferred the therapeutic composition comprises particles as described above wherein at least 80%, preferably 90 to 100% of the particles is acoustically activated upon application of ultrasound at a mechanical index, defined as the peak negative pressure divided by the square root of the frequency, of at most 3, more preferably below 2.

Generally this implies that at least 80% of the particles on application of ultrasound releases the gas and further ingredients from the core. It is highly desired that this release is taking place within a short time frame and within a small mechanical index range.

This acoustic activation can be monitored by the event count set up that is described in the examples. In this set up an activation event is qualified and counted when the amplitude of a received scattered signal (from an activated microparticle) is more than twice the noise level of the detection system.

In an exemplary embodiment, the invention relates to a therapeutic composition comprising particles comprising a gas core and polymeric shell, wherein at least 80% of the particles are activated by ultrasound energy, in a mechanical index window of 0.5 units, preferably a window of 0.4 units, more preferred 0.3 units within the mechanical index range of 0.01 to 3, more preferred 0.1 to 2, more preferred 0.4 to 2.

Preferably this activation is evidenced by an increase in the event count to at least 50 under the conditions specified in the examples.

This increase in event count preferably corresponds to an increase in echo intensity to at least 1000 times the initial value within the mechanical index window and range as described above.

A standard ultrasound transducer may be used to supply ultrasound energy. This sound energy may be pulsed but for maximal triggering of drug release it is preferred that the ultrasound energy is provided in a continuous wave. The gas containing particles can be imaged using several pulses of sound under clinically accepted diagnostic power levels for patient safety.

The invention is now illustrated by the following non-limiting examples.

### Pharmaceutical Composition

Microparticles according to the invention are optionally formulated into diagnostic compositions, preferably for parenteral administration. For example, parenteral formulations advantageously contain a sterile aqueous solution or suspension of microparticles according to this invention. Various techniques for preparing suitable pharmaceutical solutions and suspensions are known in the art. Such solutions also may contain pharmaceutically acceptable buffers and, optionally, additives such as, but not limited to electrolytes (such as sodium chloride) or antioxidants. Parenteral compositions may be injected directly or mixed with one or more adjuvants customary in acoustic imaging.

Conventional excipients are pharmaceutically acceptable organic or inorganic carrier substances suitable for parenteral, enteral or topical application which do not deleteriously react with the agents. Suitable pharmaceutically acceptable adjuvants include but are not limited to water, salt solutions, alcohols, gum arabic, vegetable oils, polyethylene glycols, gelatine, lactose, amylose, magnesium stearate, talc, silicic acid, viscous paraffin, perfume oil, fatty acid monoglycerides and diglycerides, pentaerythritol fatty acid esters, hydroxy-methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colouring, flavouring and/or aromatic substances and the like which do not deleteriously react with the active compounds.

For parenteral application, particularly suitable are injectable sterile solutions, preferably oil or aqueous solutions, as well as suspensions, emulsions, or implants, including suppositories. Ampoules are convenient unit dosages. The contrast agents containing microparticles are preferably used in parenteral application, e.g., as injectable solutions.

The therapeutic compositions of this invention are used in a conventional manner in ultrasound procedures. The diagnostic compositions are administered in a sufficient amount to provide adequate visualization and or drug delivery, to a warm-blooded animal either systemically or locally to an organ or tissues to be imaged, then the animal is subjected to the procedure. Such doses may vary widely, depending upon the diagnostic technique employed as well as the organ to be imaged.

### EXAMPLES

### Preparation of 100% Gas Filled Microparticles with Variations of Shell Thickness

0.1 g of pLLA (M_{w} 2400 g/mol) with a fluorinated end-group. prepared as described in Chlon et al. Biomacromolecules 2009 and cyclooctane (Aldrich C109401) in a ratio of 1:8, 1:5 or 1:3 were dissolved in 0.5 g dichloromethane. 120 μl of 30 mM TrisHCl buffer pH 7.5 was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra thorax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

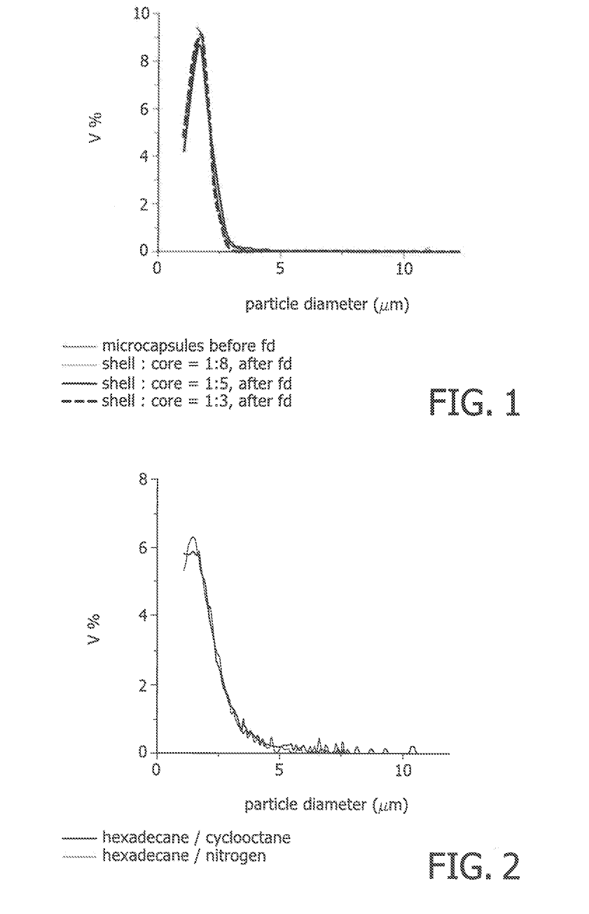

Before freeze-drying the pLLA-pFO microcaspules contain cyclooctane. After freeze-drying the nitrogen filled microparticles maintained their size distribution (Coulter counter) for all variations in shell thickness as shown in FIG. 1. Resuspending the freeze-dried microbubbles in an aqueous phase showed that they were all floating.

50% Gas-Filled pLLA-pFO Microparticles

0.0166 g of pLLA- (M_{w} 2400 g/mol) with a fluorinated end-group. prepared as described in Chlon et al. Biomacrmomolecules 2009, 0.0417 g of hexadecane (Aldrich H6703) and 0.0417 g of cyclooctane (Aldrich C109401) were dissolved in 0.5 g dichloromethane. 120 μl of 30 mM TrisHCl buffer pH 7.5 was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra thorax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C. The size distribution (Coulter counter) of the microparticles containing both hexadecane and cyclooctane was maintained after freeze-drying as shown in FIG. 2, where by means of lyophilization the cyclooctane was replaced by nitrogen, leading to half filled particles. Resuspending the freeze-dried microbubbles in an aqueous phase showed that they were all floating, indicating intact particles.

100% Gas-Filled pDLA-pFO Microparticles

0.0166 g of pDLA-pFO (M_{w} 4000 g/mol) and 0.0833 g of cyclodecane (Fluka 28699) were dissolved in 0.5 g dichloromethane. 120 μl of 30 mM TrisHCl buffer pH 7.5 was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra thorax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

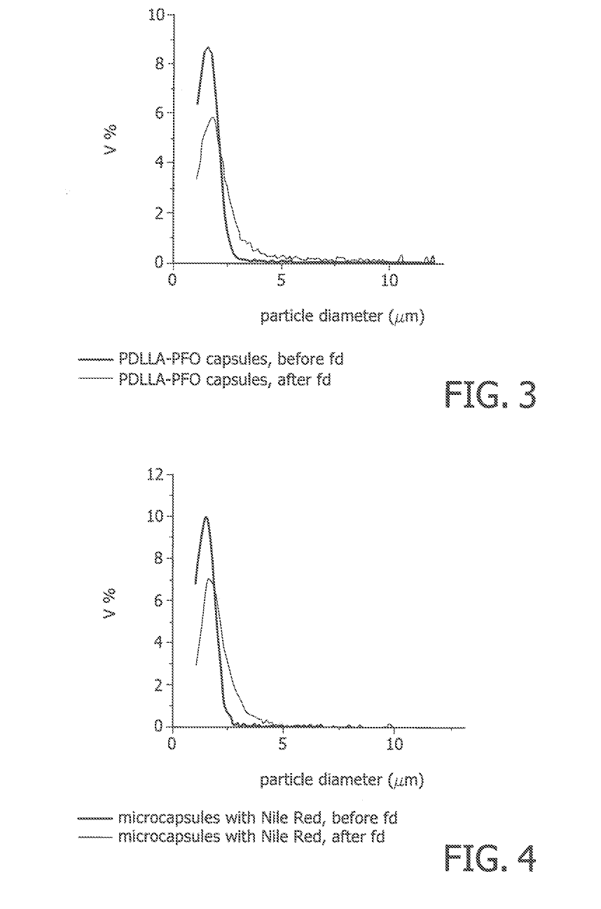

Microparticles made of amorphous pDLA-pFO showed after freeze-drying a size distribution (Coulter counter) comparable with its distribution before freeze-drying as shown in FIG. 3. Few aggregates were formed leading to a slight broadening of the size distribution peak. After resuspending these microbubbles in an aqueous phase the particles were all floating, indicating intact particles.

Microbubbles Filled with a Model Hydrophobic Molecule
100% and 50% Gas-Filled Microparticles Loaded with Sudan Black

0.0166 g pLLA-pFO (M_{w} 2400 g/mol) and 0.23 mg of Sudan Black dissolved in 0.0833 g alkane (either cyclooctane, Fluka 28699, or cyclooctane with hexadecane, Aldrich H6703, in a ratio 1:1) were dissolved in 0.5 g dichloromethane. 120 μl of milliQ water was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra thorax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

The size distributions before and after freeze-drying of the microparticles containing 100% en 50% gas-filled particles with Sudan Black were comparable and below 5 micrometers. Resuspending the freeze-dried microbubbles in an aqueous phase showed that they were all floating, indicating intact particles. Sudan Black, as a hydrophobic model compound can successfully be incorporated in microparticles prepared with a double emulsion.

The encapsulation efficiency was determined by extracting the dye from the products in dodecane measuring the absorbance gave an incorporation efficiency of 84% for 100% gas filled microbubbles and 93% for half-gas filled microbubbles. Samples made by the single emulsion method showed incorporation efficiencies of 46 and 76% for 100% gas-filled and 50% gas filled micorbubbles respectively (Kooiman et al, J. Contr. Rel. 2009)

100% Gas-Filled pLLA-pFO Microparticles with the Hydrophobic Model Compound Nile Red

0.0166 g of pLLA-pFO (M_{w} 2400 g/mol) and 0.0833 g of cyclooctane (Aldrich C109401) were dissolved in 0.5 g dichloromethane with dissolved Nile Red. 120 μl of 30 mM TrisHCl buffer pH 7.5 was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra turrax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

The size distributions (Coulter counter) before and after freeze-drying of the microparticles containing Nile Red were comparable and shown in FIG. 4. Resuspending the freeze-dried microbubbles in an aqueous phase showed that they were all floating, indicating intact particles. Nile Red, as a hydrophobic model compound can successfully be incorporated in microparticles prepared with a double emulsion. Fluorescent microscopy shows incorporation of nile red in the shell

100% and 50% Gas-Filled pLLA-pFO Microparticles Loaded with Taxol

0.0166 g of pLLA-pFO (M_{w} 2400 g/mol) and 0.1 g of alkane (either cyclooctane (Aldrich C109401), or cyclooctane with hexadecane, Aldrich H6703, in a ratio 1:1) were dissolved in 0.5 g 0.6% taxol solution in dichloromethane. 120 μl of 30 mM TrisHCl buffer pH 7.5 or pH 8.0 was added and sonicated at room temperature two times 3 seconds (1 second interval) at 110 W. To this first emulsion 2 ml of 9% polyvinyl alcohol (pVA, MW 13.000-23.000, Aldrich 363170) was added and homogenized using ultra thorax at 25.000 rpm at room temperature. The double emulsion was added dropwise to 8 ml 9% pVA agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

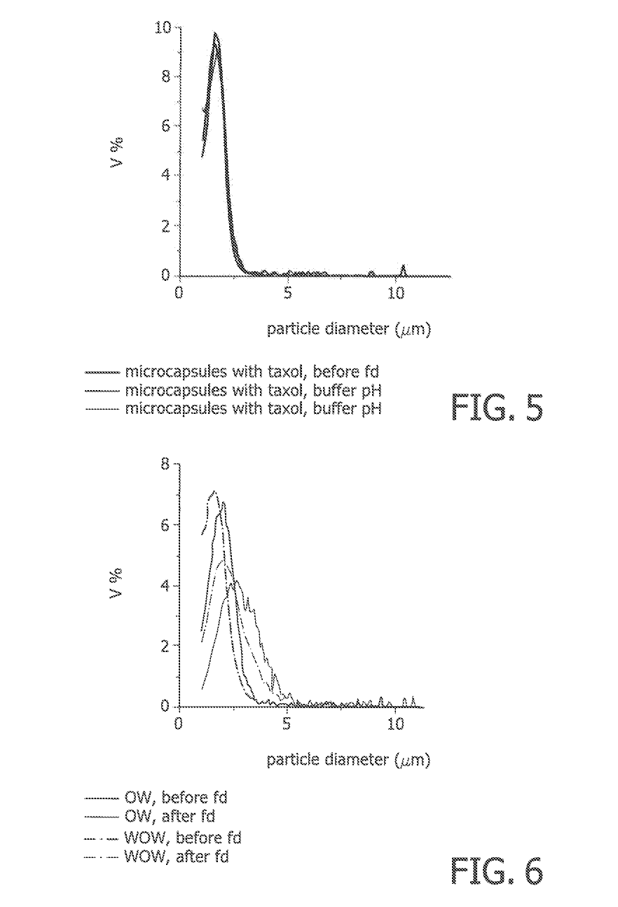

FIG. 5 shows the size distributions (Coulter counter) for the 100% and 50% gas-filled particles made by the double emulsion technique both size distributions were in the range of 1-5 μm before freeze-drying. 50% gas-filled particles showed some aggregation which is well known for the particles with residual oil. The pH of the used buffer was not of any influence on the size distribution.

Particles made by a single emulsion technique showed a same trend in size distribution before and after freeze-drying, although particle made by the single emulsion method were in general slightly larger in size. This is shown in figure

After resuspending the 100% and 50% gas-filled particles, processed by either a single or double emulsion, in an aqueous phase, they all started to float, indicating intact particles.

Paclitaxel concentrations were determined by pevered phase liquid chromatography.

10 and 20 μL aliquots in dimethylformamide of all samples were separated using reversed phase liquid chromatography (RP-LC) on an Agilent 1200 HPLC system, consisting of a binary pump, a temperature-controlled well plate sampler and a diode array detector, equipped with a Phenyl-hexyl (4.6*100 mm, 3.5 μm particles) column applying a 20 minute linear gradient of B (0.1% FA in ACN) in A (0.1% FA in water) at a flow rate of 0.7 mL/min.

Eluting compounds were subsequently analysed using UV detection at 254 nm and an Agilent ESI-ion trap (MSD) mass spectrometer capable of performing tandem mass spectrometry measuring in the alternating (switching between positive and negative) mode in the mass range m/z 200-2000.

The resulting encapsulation efficiencies were given in Table I, as a reference the incorporation efficiency in single emulsion microparticles, as described in Kooiman et al. J. Controlled Release 2009, is given.

TABLE 1
Taxol loading efficiency for microparticles
prepared by single or double emulsion

| pLLA-pFO particles | Taxol loading efficiency |
|---|---|
| Single emulsion | |
| 50% gas-filled | 15% |
| Double emulsion | |
| 100% gas-filled, buffer pH 7.5 | 21% |
| 100% gas-filled, buffer pH 8.0 | 21% |
| 50% gas-filled, buffer pH 7.5 | 39% |
| 50% gas-filled, buffer pH 8.0 | 58% |

Microparticles prepared with a double emulsion technique showed much higher paclitaxel loading efficiencies than for particles prepared with the single emulsion method. As discussed before regarding the double emulsion, the taxol crystallized not only in the (outer) aqueous phase during particle formation, but crystallization also took place to a significant extent on the surface of the encapsulated water, leading to a more efficient taxol encapsulation. The taxol loading efficiency for 50% gas-filled particles increased from 15% to 39% when prepared by a double emulsion. Increasing the pH of the buffered solution to 8.0 increased the loading efficiency even further to 58%.

For contrast agents with drug delivery from the vasculature it is preferred to inject microbubbles consisting of no additional alkane, like hexadecane. Although hexadecane is not able to keep the taxol dissolved in the capsule, introduction of this oil besides cyclooctane significantly increased the taxol encapsulation efficiency. Even when no hexadecane is incorporated the 100% gas-filled particles still showed better encapsulation results than for the 50% gas-filled microbubbles prepared with a single emulsion.

100% Gas-Filled pLLA-pFO Microparticles with the Hydrophilic Model Compound Dextran FITC

0.0166 g of pLLA-pFO(M_{w} 2400 g/mol) and 0.0833 g of cyclooctane (Fluka 28699) were dissolved in 0.5 g dichloromethane. 120 μl of 4 mg/ml Dextran-FITC pH 4.0 was added and sonicated at room temperature two times 3 seconds (1 second interval) at 110 W. To this first emulsion 2 ml of 9% polyvinyl alcohol (pVA, MW 13.000-23.000, Aldrich 363170) pH 4.0 was added and homogenized using ultra thorax at 25.000 rpm at room temperature. The double emulsion was added dropwise to 8 ml 9% pVA at pH4.0 agitated using a magnetic stirrer at 660 rpm. After stirring for 3 hours at room temperature to remove the DCM the sample was centrifuged at 4000 rpm (G is about 1720 g) for 45 minutes. The top fraction was retrieved and washed for two more times for 20 minutes with milliQ water. The sample was rapidly frozen at −80° C. in a pre-cooled vial. Freeze-drying took place using a Christ epsilon 2-6 freeze-drier for 24 hours. After freeze-drying the system was filled with nitrogen. Samples were stored at 4° C.

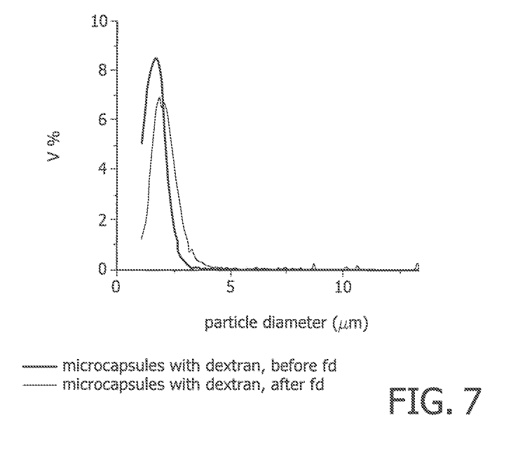

The size distributions (Coulter counter) before and after freeze-drying of the microparticles containing dextran FITC were comparable and shown in FIG. 7. Resuspending the freeze-dried microbubbles in an aqueous phase showed that they were all floating, indicating intact particles. Dextran, as a hydrophilic model compound can successfully be incorporated in microparticles prepared with a double emulsion.

By measuring the fluorescence in the supernatant the incorporation efficiency was established to be 43%. Fluorescence microscopy demonstrates the presence in the shell.

1. A polymer microparticle with a microparticle size ranging between 0.5 and 5 μm comprising a biologically active agent prepared by:
a) providing a first emulsion (A) by mixing an organic solvent (1), a biodegradable polyester, and an organic non-solvent for the polymer (2), wherein the ratio of biodegradable polyester/organic non-solvent (2) is 1:10 to 1:1% w/w, and adding to this mixture up to 40% v/v of an aqueous solution and wherein a biologically active agent is added to the organic mixture and or aqueous solution;
b) preparing a second emulsion (B) by adding to this first emulsion (A) excess of an aqueous solution;
c) applying conditions for volatizing the organic solvent (1);
d) applying conditions for removal of water; and
e) applying conditions for removal of the non-solvent (2).

2. A polymer microparticle according to claim 1, wherein the biodegradable polyester has a molecular weight between 1.000 and 200.000 g/mol.

3. A polymer microparticle according to claim 1, wherein the the ratio biodegradable polyester/organic non solvent (2) is 1:8 to 1:3

4. A polymer microparticle according to claim 1, wherein a non solvent (3) that is not removed in step e) is added to step a).

5. A polymer microparticle according to claim 1 wherein the polymer is selected from the group comprising polylactide either in the L or DL form, poly-lactide-co-glycolide, polycaprolacton, a combination thereof, or a block co-polymer thereof.

6. A polymer microparticle according to claim 5, wherein the polymer comprises at least one moiety modified with at least one hydrophobic group that is preferably selected from the group comprising fluoride, alkyl chain comprising from 6 to 24 carbon atoms or a combination of these.

7. A polymer microparticle according to claim 1, wherein non-solvent is selected from the group comprising linear or circular hydrocarbons comprising a carbon chain length of from 6 to 14 carbon atoms.

8. A polymer microparticle according to claim 7, wherein the non-solvent is selected from the group comprising cyclooctane, cyclodecane, decane, or a combination thereof.

9. A polymer microparticle according to claim 1, wherein the biologically active agent is hydrophilic.

10. A polymer microparticle according to claim 1 wherein the biologically active agent is hydrophobic.

11. A pharmaceutical composition comprising the polymer microparticles according to claim 1.

12. A polymer microparticle with a microparticle size ranging between 0.5 and 5 μm comprising a biologically active agent prepared by:
a) providing a first emulsion (A) by mixing an organic solvent (1), a biodegradable polyester, and an organic non-solvent for the biodegradable polyester (2), wherein the ratio biodegradable polyester/organic non solvent is 1:10 to 1:1, and adding to this mixture from above 0 to 40% v/v of an aqueous solution and wherein a biologically active agent is added to the organic mixture and or aqueous solution, wherein the biodegradable polyester is selected from the group comprising polylactide either in the L or DL form, poly-lactide-co-glycolide, polycaprolacton, a combination thereof, or a block co-polymer thereof, the organic solvent (1) is a good solvent for the biodegradable polyester and organic non-solvent (2) is a bad solvent for the biodegradable polyester;
b) preparing a second emulsion (B) by adding to this first emulsion (A) excess of an aqueous solution
c) applying conditions for volatizing the organic solvent (1)
d) isolating the microparticles from the aqueous phase
e) applying conditions for removal of water
f) applying conditions for removing of the non-solvent (2).

13. A polymer microparticle in accordance with claim 16, wherein steps e) and f) are performed separately.

14. A pharmaceutical composition comprising the polymer microparticles according to claim 12.
